(19) United States
(12) Patent Application Publication
(10) Pub. No.: US 2025/0185921 A1
(43) Pub. Date: Jun. 12, 2025

(54) SYSTEM AND METHOD UTILIZING OPTICAL DEPTH SENSOR FOR RECOVERING CARDIAC PULSE FROM CHEST MOTION IN DEPTH VIDEOS

(71) Applicants: Daniel W. Bliss, Phoenix, AZ (US); Yu Rong, Tempe, AZ (US)

(72) Inventors: Daniel W. Bliss, Phoenix, AZ (US); Yu Rong, Tempe, AZ (US)

(73) Assignee: Arizona Board of Regents on behalf of Arizona State University, Scottsdale, AZ (US)

(21) Appl. No.: 18/973,975

(22) Filed: Dec. 9, 2024

Related U.S. Application Data
(60) Provisional application No. 63/607,544, filed on Dec. 7, 2023.

(51) Int. Cl. A61B 5/0205; A61B 5/00; A61B 5/024; G06V 10/25; G06V 10/26; G06V 10/36; G06V 20/40; G06V 40/10

(52) U.S. Cl. CPC A61B 5/0205; A61B 5/02416; A61B 5/7203; G06V 10/25; G06V 10/26; G06V 10/36; G06V 20/49; G06V 40/15; G06V 2201/03

(57) ABSTRACT

A system and method utilizes optical depth sensors to estimate heart rate of one or more subjects. A method includes obtaining optical depth video data of at least one subject; identifying a region of interest of the subject(s) from optical depth video data; segmenting the region of interest into multiple areas; identifying pixel intensity with respect to time in the areas to produce a depth signal data matrix including multiple spatial channels; decomposing the depth signal data matrix into a low-rank spatial-temporal eigenvector matrix to produce refined depth signal data streams; and projecting the refined depth signal data streams onto a selected pulsatile direction and producing a cardiac pulse signal for the subject(s).

### CROSS-REFERENCE TO RELATED APPLICATION(S)

This application claims priority to U.S. Provisional Patent Application No. 63/607,544 filed on Dec. 7, 2023, wherein the entire contents of the foregoing application are hereby incorporated by reference herein.

### TECHNICAL FIELD

This disclosure relates to vital sign detection, and more specifically to optical detection of cardiac pulse signals.

### BACKGROUND

Cardiovascular diseases are a leading cause of death worldwide. Continuous heart health monitoring is promising as preventing healthcare for heart failure. Conventional monitoring technologies include electrocardiogram (ECG) and photoplethysmogram (PPG) technologies as provided by various smart wearable devices such as Apple® Watch and Fitbit® devices. ECG captures the electric heart signals utilizing electrical nodes attached to a human body, while PPG detects blood volume changes due to heart contraction and retraction by placing an optical sensor on human skin. Either method requires direct physical contact that might cause discomfort for certain groups of people.

On the other hand, non-contact remote heart health monitoring has attracted significant attentions from the research community. These methods mainly rely on radio frequency (RF) sensors and optical sensors. An optical approach using light waves, such as visible light and infrared (IR), has been considered most promising. Two major measurement principles for estimating cardiac pulses using optics are color based methods and motion based method. Among color based methods, recorded videos can capture color changes due to blood flow when the exposed skin is illuminated (e.g., by ambient light or other light sources) according to an approach known as remote imaging photoplethysmography (RIPPG). Among motion-based methods, cardiac pulses may be estimated from human body videos by measuring the ballistic forces of the heart caused by the sudden ejection of blood into the great vessels with each heartbeat, often referred to as ballistocardiography (BCG).

The art continues to seek improvement in recovering heart rate information utilizing non-contact optical approaches including BCG, to permit such approaches to be automated and to overcome challenges such as addressing unwanted motion interference that may be attributable to breathing and involuntary body motion.

### SUMMARY

Aspects of the present disclosure relate to a novel approach for extracting cardiac pulses using optical depth sensors, such as depth cameras that derive depth images based on time-of-flight (ToF) technology to extract ballistocardiography (BCG) signals and estimate heart rate of one or more subjects.

In one aspect, the disclosure relates to a method for remotely monitoring heart rate of at least one subject, the method comprising: obtaining optical depth video data of the at least one subject; identifying a region of interest of the at least one subject from the optical depth video data; segmenting the region of interest into multiple areas; identifying pixel intensity with respect to time in the multiple areas to produce a depth signal data matrix including multiple spatial channels; decomposing the depth signal data matrix into a low-rank spatial-temporal eigenvector matrix to produce refined depth signal data streams; and projecting the refined depth signal data streams onto a selected pulsatile direction and producing a first cardiac pulse signal for the at least one subject.

In certain embodiments, the selected pulsatile direction comprises an optimum pulsatile direction at which cross power spectral density, comprising spectral coherence values as a function of frequency, is maximized.

In certain embodiments, the method further comprises applying bandpass temporal filtering to eliminate excessively high frequency and excessively low frequency data to reduce noise in the first cardiac pulse signal.

In certain embodiments, the eigenvector matrix comprises data representing magnitude and direction of each spatial channel contributing to eigenvectors of the eigenvector matrix.

In certain embodiments, the method further comprises detecting a torso area of the at least one subject in the optical depth video data, wherein the identifying of the region of interest comprises removing edge areas from the detected torso area.

In certain embodiments, the method further comprises detecting a head area of the at least one subject in the optical depth video data, wherein the identifying of the region of interest comprises removing edge areas from the detected head area.

In certain embodiments, the method further comprises detecting a respiration rate of the at least one subject.

In certain embodiments, the at least one subject comprises a plurality of subjects, and the method comprises producing a different first cardiac pulse signal for each subject of the plurality of subjects.

In another aspect, the disclosure relates to a heart rate monitoring system comprising: an optical depth sensor; and an image processor configured to: receive optical depth video data of at least one subject; identify a region of interest of the at least one subject from the optical depth video data; segment the region of interest into multiple areas; identify pixel intensity with respect to time in the multiple areas to produce a depth signal data matrix including multiple spatial channels; decompose the depth signal data matrix into a low-rank spatial-temporal eigenvector matrix to produce refined depth signal data streams; and project the refined depth signal data streams onto a selected pulsatile direction and produce a first cardiac pulse signal for the at least one subject.

In another aspect, the disclosure relates to a non-transitory computer readable medium comprising computer-readable instructions, that when executed by a processor, cause the processor to perform operations, the operations comprising: identifying a region of interest of at least one subject from optical depth video data of the at least one subject; segmenting the region of interest into multiple areas; identifying pixel intensity with respect to time in the multiple areas to produce a depth signal data matrix including multiple spatial channels; decomposing the depth signal data matrix into a low-rank spatial-temporal eigenvector matrix to produce refined depth signal data streams; and projecting the refined depth signal data streams onto a selected pulsatile direction and producing a first cardiac pulse signal for the at least one subject.

In another aspect, any two or more features of aspects and/or embodiments disclosed herein may be combined for additional advantage.

### BRIEF DESCRIPTION OF DRAWINGS

FIG. 1 is a schematic diagram of an exemplary heart rate monitoring system that remotely detects heart rate of one or more subjects, using an optical depth sensor and an image processor, according to methods described herein.

FIG. 2A is a grayscale converted digital photograph of a subject originally obtained by RGB imaging.

FIG. 2B is a grayscale converted digital photograph of a subject originally obtained by NIR imaging.

FIG. 2C is a grayscale converted digital photograph of a subject originally obtained by depth imaging.

FIG. 3A provides an illustration of a simulated motion generator as well as plots (waveforms) of motion of the simulated motion generator (with a length of 3 cm and a step size of 0.1 cm) observed by RGB detection, NIR detection, and depth detection, as well as a truth plot derived from an input signal to the simulated motion generator.

FIG. 3B provides an illustration of a human subject wearing a respiratory belt, as well as plots (waveforms) of chest motion of the human subject observed by RGB detection, NIR detection, and depth detection, as well as a truth plot obtained from the respiratory belt worn by the human subject.

FIGS. 4A-4D in combination represent a high-level processing diagram outlining steps of a method for extracting a pulse signal from chest depth video according to one embodiment, wherein FIG. 4A illustrates torso landmarks estimation and tracking by applying pose estimator on depth images (at left) and torso ROI selection and segmentation for effective use of depth pixels (at right), FIG. 4B shows construction of a desired low-rank spatial-temporal matrix by removing other motion noise as outliers, FIG. 4C shows projection onto the most pulsatile direction and pulse signal selection in eigen space based on known spectral features to perform pulse extraction, and FIG. 4D shows signal enhancement by applying a narrow spectral filter for suppressing out-of-band noise.

FIG. 5 is an overlay plot of cross power spectral density (CPSD) estimate including examples from a (best) pulsatile spatial channel and a less (poor) pulsatile spatial channel, showing maximum coherence peaks at the heartbeat frequency for the pulsatile channel, and showing small coherence estimates (near zero) obtained from the poorly pulsatile channel at the same heartbeat frequency.

FIG. 6A is a grayscale-converted graphical eigenvector plot for multiple spatial channels for an undesired motion component overlaid on a depth image frame, with grayscale shading representing magnitude of motion contributing to the eigenvectors, and with dots and triangular markers denoting opposite motion directions (inward/outward) represented by signs of elements in each eigenvector.

FIG. 6B is a grayscale-converted graphical eigenvector plot for multiple spatial channels including a selected eigenvector (within a superimposed oval) representing a cardiac pulse, overlaid on a depth image frame, with grayscale shading representing magnitude of motion contributing to the eigenvectors, and with dots and triangular markers denoting opposite motion directions (inward/outward) represented by signs of elements in each eigenvector.

FIG. 6C is a grayscale-converted graphical eigenvector correlation plot for multiple spatial channels derived from the plot of FIG. 6B, with grayscale shading representing the correlation magnitude, and with a superimposed oval surrounding a region with highest correlation corresponding to a point of maximal impulse.

FIG. 6D is a cross power spectral density (CPSD) plot of relative magnitude versus heart rate derived from depth signal data based on the graphical eigenvector plot of FIG. 6A overlaid with a reference PPG spectrum, showing poor correlation between the depth signal data peaks and PPG peaks.

FIG. 6E is a cross power spectral density (CPSD) plot of relative magnitude versus heart rate derived from depth signal data based on the graphical eigenvector plot of FIG. 6B overlaid with a reference PPG spectrum, showing high correlation between the depth signal data peaks and PPG peaks.

FIG. 6F is an image of a portion of a human skeleton overlaid with a marking showing the location on a subject's torso at which cross power spectral density is maximized.

FIG. 7(a.1) to 7(e.1) provide plots of inter-beat-interval for five different subjects obtained by remote optical depth video processing according to a method disclosed herein.

FIGS. 7(a.2) to 7(e.2) provide plots of inter-beat-interval for the same five subjects as represented in FIGS. 7(a.1) to 7(e.1), but obtained by a conventional photoplethysmography (PPG) method.

FIG. 8 is a table summarizing measurement statistics including heart rate accuracy, heart rate absolute error, missed beat detection percentage, and spectral coherence percentage for ten subjects.

FIGS. 9A to 9E provide bar charts showing heart rate estimation accuracy for five subjects.

FIG. 10A provides a plot of estimated respiration pattern derived from depth signal data as disclosed herein for one subject.

FIG. 10B provides a plot of respiration pattern derived from an earlobe photoplethysmography (PPG) sensor for the same subject and interval as represented in FIG. 10A.

FIG. 10C provides a plot of a recovered heartbeat waveform derived from depth signal data for the same subject and interval as represented in FIGS. 10A and 10B.

FIG. 10D provides a plot of a recovered heartbeat waveform derived from the earlobe photoplethysmography (PPG) sensor for the same subject and interval as represented in FIGS. 10A-10C.

FIG. 11 provides plots for empirical distribution function versus errors for RPCA, for PCA without outlier removal, and for direct averaging of depth pixel data in a region of interest of a subject's chest.

FIG. 12A is a grayscale-converted depth image of a subject with a superimposed region of interest (ROI), obtained at a distance of 0.5 m from a depth image camera.

FIG. 12B is a grayscale-converted depth image of the same subject and superimposed region of interest (ROI) as depicted in FIG. 12A, but obtained at a distance of 2 m from a depth image camera.

FIG. 13 is a table providing heart rate estimation accuracy as a function of distance (for distance values of ranging from 0.5 to 2 meters) for tolerance levels of 5, 3, and 1 beats per minute, respectively.

FIG. 14A is a grayscale-converted depth image of three subjects, marked as left, center, and right, respectively.

FIG. 14B is a bar chart depicting heart rate estimation accuracy for each of the three subjects of FIG. 14A at tolerance levels of 3 beats per minute and 1 beat per minute.

FIG. 15 is a bar chart depicting heart rate estimation accuracy for a subject that is uncovered, a subject wearing thin clothing, and a subject wearing thick clothing at tolerance levels of 3 beats per minute and 1 beat per minute.

FIG. 16A is a cardiac waveform obtained from depth signal data obtained from the back of a head of a subject using a method disclosed herein.

FIG. 16B is a grayscale-converted depth image for an upper rear portion of a subject including the back of the head of the subject.

FIG. 16C is a cross power spectral density (CPSD) plot of relative magnitude versus heart rate derived from depth signal data obtained from the back of a head of a subject overlaid with a reference PPG spectrum, showing high correlation between the depth signal data peaks and PPG peaks.

FIG. 17 is a block diagram of at least a portion of a heart rate monitoring system according to embodiments disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Aspects of the present disclosure relate to a system and method for extracting cardiac pulses using optical depth sensors, not in contact with any subject to be monitored, to estimate heart rate of one or more subjects.

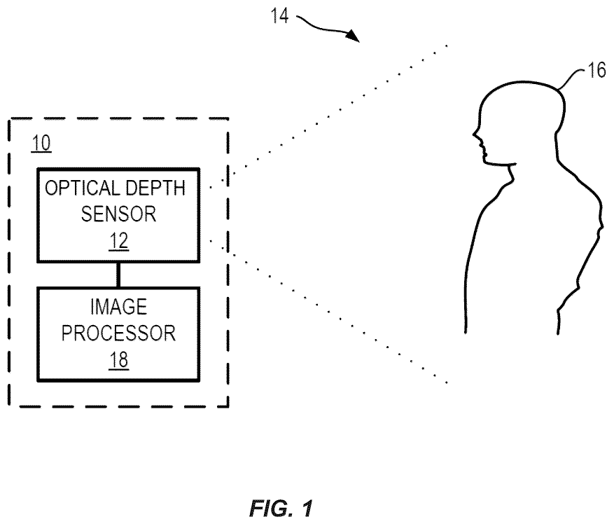

FIG. 1 is a schematic diagram of an exemplary heart rate monitoring system 10, which remotely detects heart rate of one or more subjects using an optical depth sensor 12, such as an optical depth camera, optionally embodied in or incorporating a time-of flight depth camera. The optical image sensor 12 provides video data of a nearby environment 14 including one or more subjects 16 (e.g., human subject(s)). The heart rate monitoring system 10 includes an image processor 18 configured to extract vital signs of the subject(s) 16 from the video data provided by the optical image sensor 12.

The heart rate monitoring system 10 detects, measures, and/or monitors heart rate, optionally in conjunction with respiration rate, of the subject(s) 16 based on detection of changes in displacement of skin of the subject(s) attributable to heart pumping. The approach described herein allows for a light source illuminating subjects to be ambient light, but other light sources may be used as well.

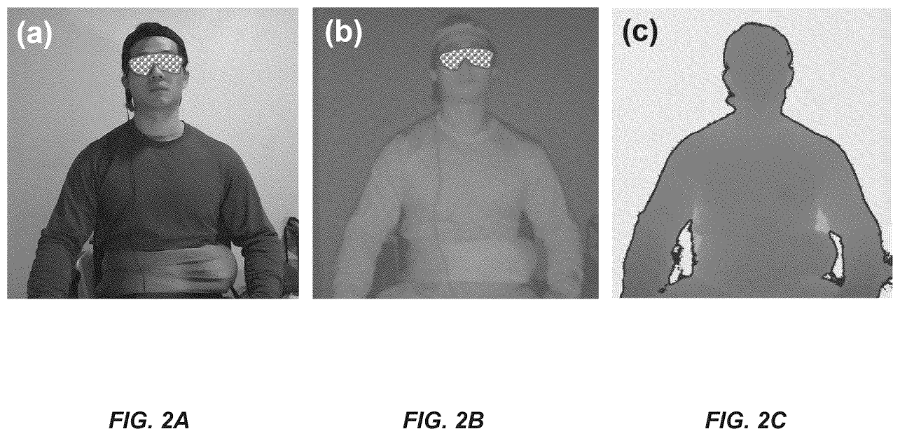

The use of depth sensors provides advantages over RGB video and near-infrared (NIR) technology. Unlike RGB video, depth video information will generally not reveal human identity. See, for example, FIGS. 2A-2C, which provide digital photographs of the same subject, but obtained by different imaging technologies-namely, FIG. 2A utilizes RGB imaging, FIG. 2B utilizes NIR imaging, and FIG. 2C utilizes depth imaging. As shown, human identity is nearly impossible to determine with depth imaging alone.

Deploying depth camera for micromotion estimation provides enjoy higher dynamic range and accuracy in reconstruction of motion as compared to the use of RGB and NIR. The raw pixel intensity in RGB, NIR and depth videos have significantly different sensitivity levels to micromotion. This difference is explained by the operation of these imaging modalities. The depth pixel intensity variation is directly related to the physical displacement, whereas the motion induced light intensity variation in RGB and NIR is weakly related to the observed motion through a highly non-linear transformation The depth accuracy also depends on the system dynamic range. The pixel intensity for RGB and NIR are commonly represented in 8-bit precision, which corresponds to 28=256 levels. For depth, it is commonly described in 16 bit precision, or 2¹⁶=65,536 levels. The depth pixel is therefore able to represent much finer values.

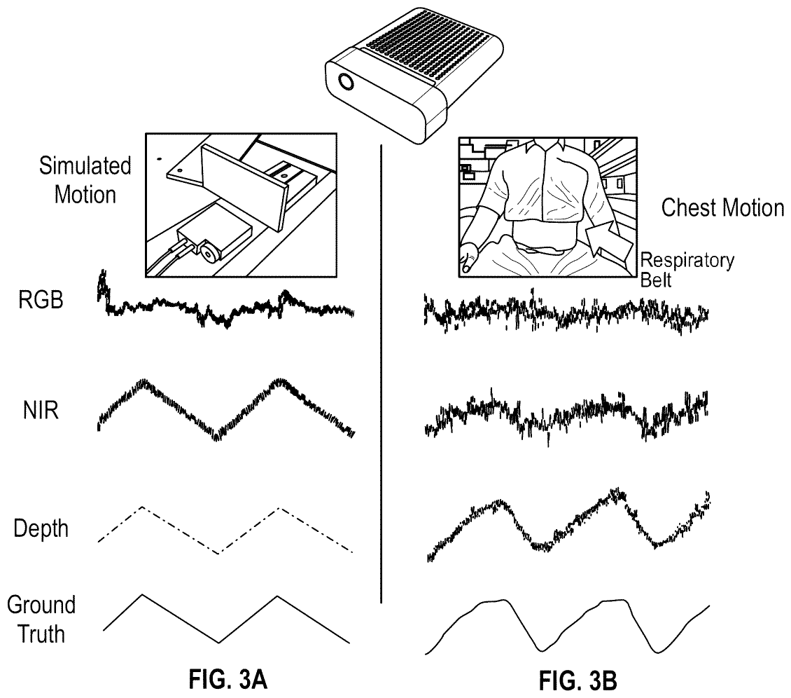

The example shown in FIGS. 3A-3B confirms the superior motion estimation performance of depth image as compared to RGB and NIR. FIG. 3A provides a digital photograph of a simulated motion generator (i.e., actuator) as well as plots (waveforms) of motion of the simulated motion generator (with a length of 3 cm and a step size of 0.1 cm) observed by RGB detection, NIR detection, and depth detection, as well as a truth plot derived from an input signal to the simulated motion generator. The actuator generated a 3-centimeter (cm) linear displacement with a step size of 0.1 cm in a back and forth fashion on a precision stage. FIG. 3B provides a digital photograph of a human subject to a simulated motion generator as well as plots (waveforms) of motion of the simulated motion generator observed by RGB detection, NIR detection, and depth detection, as well as a truth plot obtained from a respiratory belt worn by the human subject. In the cases of both heart rate and respiratory detection, the depth camera produced less noisy estimation and better reconstruction of the observed motion when compared to the counterparts and reference motion signal. In FIGS. 3A and 3B, 400 randomly picked raw pixels within the manually selected area in the image frames were averaged to generate the above result.

A preferred type of depth sensor for use with systems and methods disclosed herein includes a time-of flight depth camera. One example of such a camera is provided in an Azure Kinect developer kit, commercially available from Microsoft Corp. (Redmond, Washington, US). This kit includes an advanced depth camera (including a 1 megapixel depth sensor) and a spatial microphone array, together with an RGB camera and orientation sensor. The maximum depth image resolution is 1024×1024 with a maximum frame rate of 15 frames per second (FPS), which is sufficient to capture pulse rate. The depth image is derived based on time-of-flight (ToF) technology. Precise distance measurement in depth pixel is leveraged to extract BCG signals. The chest motion due to heartbeat ranges from 0.1 to 1 millimeter (mm) while the depth camera provides approximately 1 mm ranging accuracy at short distances of less than <2 meters (m) in a line-of-sight condition. Accordingly, the Azure Kinect may be used for short-range heartbeat detection, and methods disclosed herein enable attainment of sub-mm displacement measurement accuracy with signal processing by manipulating many depth pixels.

When a torso area is imaged as a basis for heart rate detection, a challenge is to separate out stronger chest motions due to respiration and involuntary body motions to obtain clean cardiac pulse signal. Additionally, a majority of the area of a torso contributes to the respiration signal, such that when involuntary body motion occurs, it will impact all the pixels in the template. Despite the fact that the heart chamber can be roughly located when the upper body is identified, a predefined heart geometry might not work for every subject due to variation in heart location among individuals. The inventors were therefore motivated to develop a data-driven approach to extract pulse without prior knowledge of heart location. The rationale behind this approach is that heartbeat, respiration and random body motions behave in a statistically different manner.

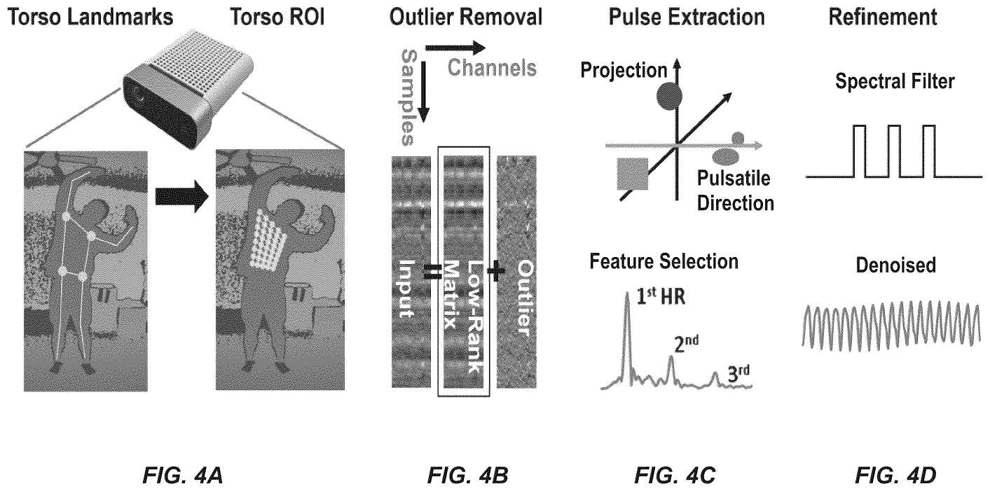

FIGS. 4A-4D in combination represent a high-level processing diagram outlining steps of a method for extracting a pulse signal from chest depth video according to one embodiment, wherein FIG. 4A illustrates torso landmarks estimation and tracking by applying pose estimator on depth images (at left) and torso ROI selection and segmentation for effective use of depth pixels (at right), FIG. 4B shows construction of a desired low-rank spatial-temporal matrix by removing other motion noise as outliers, FIG. 4C shows projection onto the most pulsatile direction and pulse signal selection in eigen space based on known spectral features to perform pulse extraction, and FIG. 4D shows signal enhancement by applying a narrow spectral filter for suppressing out-of-band noise. The various steps represented by FIGS. 4A-4B of a method for extracting a pulse signal from chest depth video according to one embodiment will now be described. One step includes obtaining optical depth video data of at least one subject, such as by using a depth camera as disclosed herein, distanced away from any subject(s) to be monitored. Another step includes identifying a region of interest of the at least one subject from the optical depth video data. In certain embodiments, a region of interest may be a subregion of a torso, or a head (particularly the back of a head), of a subject. In certain embodiments, a human torso may be automatically identified in depth videos by detecting landmarks using MediaPipe Pose (an open source framework). An area within a polygon bounded by the landmarks forms an initial torso template. Thereafter the torso template is reduced in size (shrunken) by removing edge areas to define a chest region of interest (ROI). The inventors have observed large pixel intensity variation from edge areas due to landmark tracking inconsistency.

A further step includes segmenting the ROI interest into multiple areas (e.g., a grid of multiple cells). In one embodiment, a ROI is divided into a grid of 7×4 cells (e.g., as shown in FIG. 4B, with four columns and seven rows of cells superimposed on a torso of a subject). The corresponding depth values in c^{th} cell are averaged to enhance noise performance. For each video frame t=1, . . . , T, a depth time series is denoted as p_{c}(t). Further high-level steps include outlier removal and pulse extraction, as detailed below.

### Signal Decomposition

The spatial channels over the chest ROI are a mixture of pulse, respiration and other involuntary body movements. We resort to robust principal component analysis (RPCA) to remove outliers from abrupt body motion and region tracking errors, and then projects the refined depth streams onto the optimum pulsatile direction. Given C depth streams, C-dimensional motion trajectories are provided, and a depth signal matrix D is provided according to Equation (1):

$$D = [ {d}_{1} ( t ) , … , {d}_{c} ( t ) , … , {d}_{C} ( t ) ] , \qquad ( 1 )$$

where t=1, . . . , T denotes the discrete sampled time instance and dc (t) is a column vector ∈R^{T×1}.

Robust principal component analysis (RPCA) seeks to decompose the depth signal matrix D into a structure low-rank matrix X and a sparse matrix S containing outliners, according to Equation (2):

$$D = X + S . \qquad ( 2 )$$

X is obtained by solving the following optimization problem using the alternating directions method, according to Equation (3):

$${minimize}_{X , S} {‖ X ‖}_{*} + γ {‖ S ‖}_{1} , subject to D = X + S , \qquad ( 3 )$$

where |X|* denotes the nuclear norm (sum of singular values) of the matrix X. ∥S∥₁ denotes the [symbol] ₁ norm of S. γ is a regularization parameter. The parameter γ controls the relative proportion of the signal energy X that will be absorbed into the noise component S. A smaller value of γ allows more of the signal X to be considered as noise S and vice-versa. Here the γ is empirically chosen as 0.02 since this value gives consistent heart rate estimation performance in the present study.

Eigenvalue decomposition operates on the spatial temporal covariance matrix B=X^{T}X and finds the directions of maximum statistical variance as the eigenvectors V of B, according to Equation (4)

$$BV = V Σ , \qquad ( 4 )$$

where V=[v₁, . . . , v_{C}] denotes the eigenvector matrix with each column as eigenvector and ∈=Diag[λ₁, . . . . λ_{C}] denotes the diagonal matrix with the corresponding eigenvalues Δ_{c} on its diagonal. The sign of the elements in eigenvector represents the C-dimensional direction while the absolute value of the element represents the motion strength.

Projection of the mean-centered spatial temporal matrix X^{⋅} onto the optimal pulsatile direction, represented by a selected direction v_{c}, produces the cardiac pulse signal p_{c}(t), according to Equation (5):

$${p}_{c} ( t ) = \dot{X} {v}_{c} . \qquad ( 5 )$$

The eigenvectors are ordered based on eigenvalues, which indicate the statistical variance in the C-principal axes, determined by the eigenvectors. The estimated pulse signal p_{c}(t) is obtained by selecting a proper eigenvector that combines the columns of \dot{X} streams constructively. Though λ₁ explains most of the variance in the data, p₁(t) might not be the best choice and often it contains residual breathing signal. Our consideration limits to top five eigenvectors because majority information is carried in the first a few eigenvalues. The best possible v_{c} and p_{c}(t) are determined by checking their spectral peaks including the fundamental heartbeat frequency as well as 2^{nd} and 3^{rd} order harmonics.

For denoising the RPCA output, the spectral filter design narrows around the estimated HR as well as the 2nd and the 3rd order harmonics to reduce noise. This recognizes that a typical PPG signal spectrum has a sharp dominant peak and another two harmonic peaks. The spectral filter Y is represented according to Equation (6):

$$Υ ( f ) = \{ \begin{matrix}1 , & {f}_{HR} - \frac{ω}{2} \leq f \leq {f}_{HR} + \frac{ω}{2} \\ 1 , & 2 {f}_{HR} - \frac{ω}{2} \leq f \leq 2 {f}_{HR} + \frac{ω}{2} \\ 1 , & 3 {f}_{HR} - \frac{ω}{2} \leq f \leq 3 {f}_{HR} + \frac{ω}{2} \\ 0 , & elsewhere\end{matrix} , \qquad ( 6 )$$

where the sub-band window size w is chosen empirically as 0.25 Hz or 15 beats to ensure covering HR variability within the processing window. Given a 15-second processing window, the frequency resolution is about 0.067 second or 4 beats per resolution bin. The filter is constructed in frequency domain and multiplied with the spectrum of p_{c}(t).

The denoised pulse signal is obtained via inverse Fourier transform (iFFT) according to Equation (7):

$${p}_{d} ( t ) = iFFT [ {p}_{c} ( f ) ⊙ Υ ( f ) ] , \qquad ( 7 )$$

where ⊙ denotes element-wise production.

### Interpreting Cardiac Pulse Spatial Distribution Over Chest ROI

A procedure to infer how the eigenvector (projection operator) combines depth streams to extract pulses is now provided. The pulse correlation map indicates the cardiac pulse distribution over the chest ROI. In this study, the correlation value is calculated as the spectral coherence between the reference PGG signal and the depth streams X(:,c), at the c^{th} spatial channel evaluated at the reference HR.

The spectral coherence shows better noise resilience compared to the temporal coherence since the time domain signals are highly distorted. Cross-correlation function R_{1,2}(T) computes the reference PPG r₁(t) and the analyzing signal r₂(t) according to Equation (8):

$${R}_{1 , 2} ( τ ) = {lim}_{{T}_{c} \to ∞} \frac{1}{{T}_{c}} {\int }_{0}^{{T}_{c}} dt {r}_{1} ( t ) {r}_{2} ( t - τ ) , \qquad ( 8 )$$

where Tc denotes the coherence analyzing interval. The spectral coherence, S_{1,2}(f), is defined as the FT of R_{1,2}(T), according to Equation (9):

$${S}_{1 , 2} ( f ) = F \{ {R}_{1 , 2} ( τ ) \} = {\int }_{- ∞}^{∞} d τ {R}_{1 , 2} ( τ ) {e}^{- j 2 π f τ} . \qquad ( 9 )$$

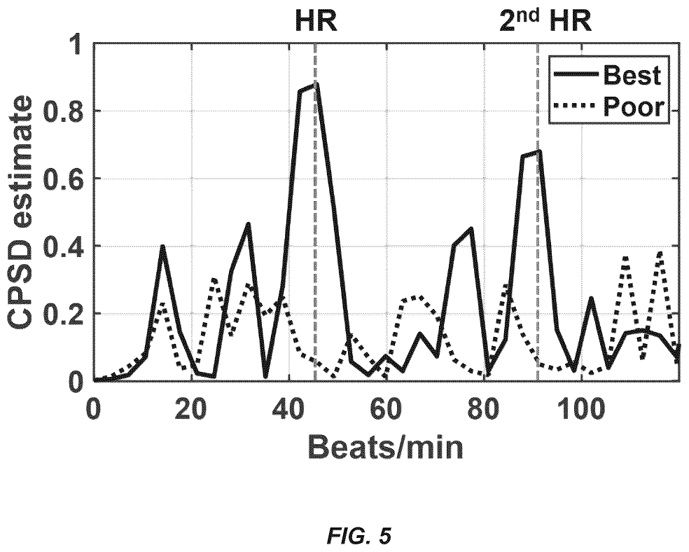

By plotting spectral coherence values as a function of frequency, it generates cross power spectral density (CPSD) spectrum. Any correlation between the signals reflects on the CPSD spectrum. If the signals are uncorrelated, the CPSD spectrum is zero or close to zero at all frequencies for uncorrelated signals. When the two signals are highly correlated, the CPSD spectrum will exhibit peaks at the common frequency locations, as shown in FIG. 5.

FIG. 5 is an overlay plot of cross power spectral density (CPSD) estimate including examples from a (best) pulsatile spatial channel and a less (poor) pulsatile spatial channel, showing maximum coherence peaks at the heartbeat frequency for the pulsatile channel, and showing small coherence estimates (near zero) obtained from the poorly pulsatile channel at the same heartbeat frequency.

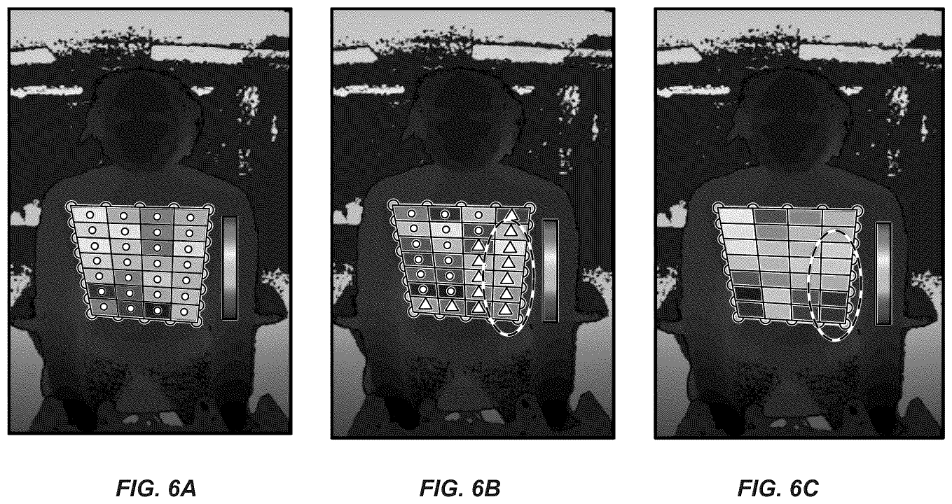

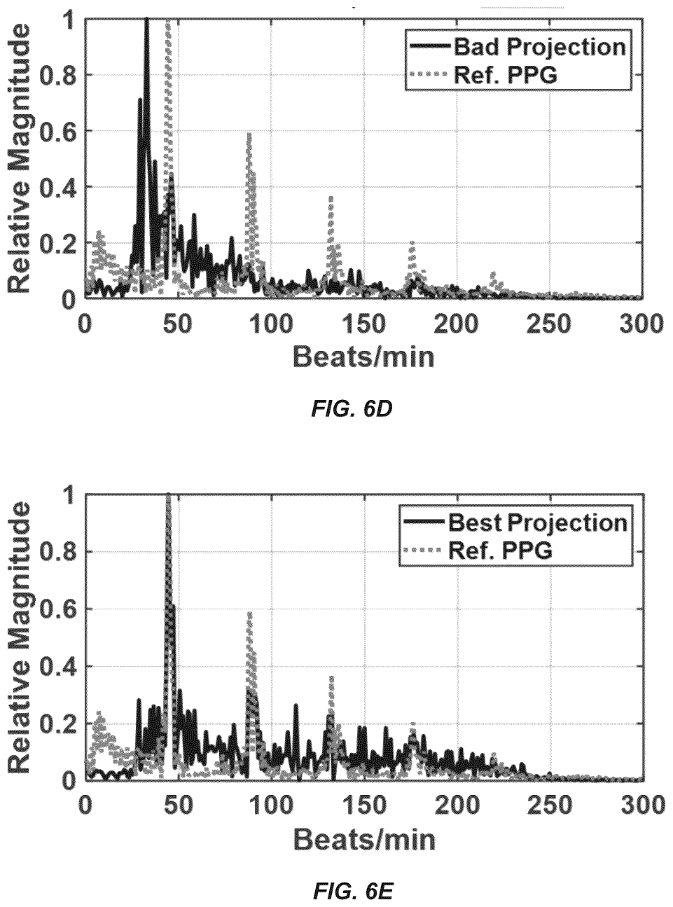

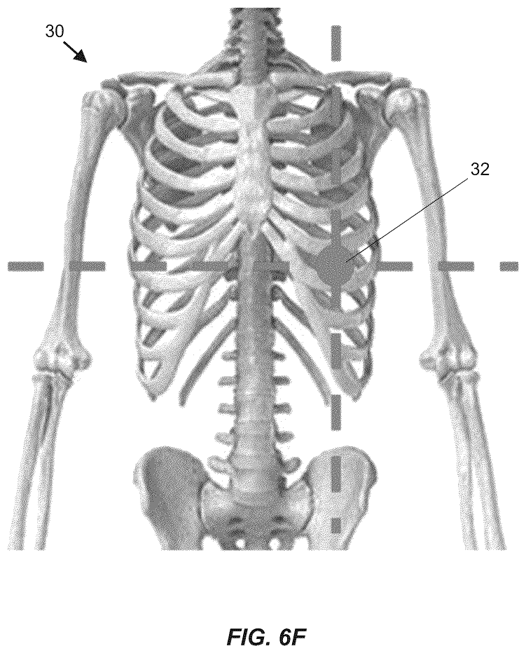

FIGS. 6A-6B provide grayscale-converted graphical eigenvector plots, and FIG. 6C is a grayscale-converted graphical eigenvector correlation plot, to help illustrate the effectiveness of signal decomposition in finding the best pulsatile direction in the presence of motion interference. Each eigenvector represents C-dimensional direction and strength of motion in C spatial locations, in which the markers denote the two movement direction inward/outward with respect to the imaging plane. In FIGS. 6A and 6B, grayscale shading represents magnitude of motion contributing to the eigenvectors, and with dots and triangular markers denoting opposite motion directions (inward/outward) represented by signs of elements in each eigenvector.

FIG. 6A is a graphical eigenvector plot for multiple spatial channels for an undesired motion component overlaid on a depth image frame. The undesirable eigenvector plot of FIG. 6A implies that little pulsatile information is contained in the corresponding principal component. FIG. 6D is a cross power spectral density (CPSD) plot of relative magnitude versus heart rate derived from depth signal data based on the graphical eigenvector plot of FIG. 6A overlaid with a reference PPG spectrum, showing poor correlation between the depth signal data peaks and PPG peaks.

According to methods herein, the chosen eigenvector projects the spatial depth streams onto the best pulsatile principal axis. FIG. 6B is a graphical eigenvector plot for multiple spatial channels including a selected eigenvector (within a superimposed oval) representing a cardiac pulse, overlaid on a depth image frame. FIG. 6E is a cross power spectral density (CPSD) plot of relative magnitude versus heart rate derived from depth signal data based on the graphical eigenvector plot of FIG. 6B overlaid with a reference PPG spectrum, showing high correlation between the depth signal data peaks and PPG peaks, but with additional noise in the signal derived from depth signal data. FIG. 6B shows that the desired project map has stronger magnitude values (motion strength) in the lower-right corner with the triangular markers (motion direction), as highlighted by the superimposed oval marker at the lower-right corner area. According to the basic physiological science, these locations correspond to the point of maximal impulse of normal subjects as displayed in FIG. 6F, representing a portion of a human skeleton 30 overlaid with a marking 32 showing the location on a subject's torso at which cross power spectral density is maximized.

FIG. 4D depicts spectral filtering to obtain a denoised waveform. Temporal filtering focuses on data at the appropriate frequency range of interest. The commonly used frequency range is 50 to 100 beats/min (BPM) since the normal rest heart rate (HR) is about 1 Hz (or 60 BPM). However, depending on gender, physical condition and fitness level, the resting HR can be lower than 40 BPM and can be higher than 100 BPM. Thus, an expanded temporal filter is considered, in a range of 30 to 240 BPM. The upper limit of the filter is further expanded since HR harmonics aid in identifying the fundamental HR. The lower cut-off frequency helps recovery of low resting HR at the cost of introducing more motion leakage from other frequency bands. Restated, bandpass temporal filtering may be applied to eliminate excessively high frequency and excessively low frequency data to reduce noise in a cardiac pulse signal.

To summarize various items above, a computer vision model may be pre-trained on depth images to track human torso landmarks and create a chest region of interest. Then cardiac pulse information may be extracted by applying robust principal component analysis to find the most pulsatile signal. A representation in eigen space was developed for signal decomposition and selection.

Consistent with the foregoing, a method for remotely monitoring heart rate of at least one subject comprises: obtaining optical depth video data of the at least one subject; identifying a region of interest of the at least one subject from the optical depth video data; segmenting the region of interest into multiple areas; identifying pixel intensity with respect to time in the multiple areas to produce a depth signal data matrix including multiple spatial channels; decomposing the depth signal data matrix into a low-rank spatial-temporal eigenvector matrix to produce refined depth signal data streams; and projecting the refined depth signal data streams onto a selected pulsatile direction and producing a first cardiac pulse signal for the at least one subject. The method may further comprise applying bandpass temporal filtering to eliminate excessively high frequency and excessively low frequency data to reduce noise in the first cardiac pulse signa. In certain embodiments, the selected pulsatile direction comprises an optimum pulsatile direction at which cross power spectral density, comprising spectral coherence values as a function of frequency, is maximized. In certain embodiments, the eigenvector matrix comprises data representing magnitude and direction of each spatial channel contributing to eigenvectors of the eigenvector matrix.

### Experiments

Experimentation was performed to evaluate motion based methods for detecting cardiac pulse detection using depth videos as described herein. Ten volunteers (seven (7) males and three (3) female subjects of ages between 20 and 35) participated in an experimental study. RGB, NIR and depth videos were recorded simultaneously and synchronized with other reference sensors. Each test subject was seated in a steady chair and instructed to remain still with normal breathing during the recording. For regular recording, the distance from the recording system and the subjects was approximately 1 meter. However, for range testing, the distance from the subject to the depth camera varied. Each subject was recorded for four minutes in total. The reference PPG signals were taken from an oximeter. Specific experiments were conducted to validate methods herein, including inter-beat-interval (IBI) analysis for subject specific HR dynamics, quantitative evaluation of HR estimation, discussion of algorithm performance, study of environment issues, and generalization testing.

### 1) Inter-Beat-Interval (IBI):

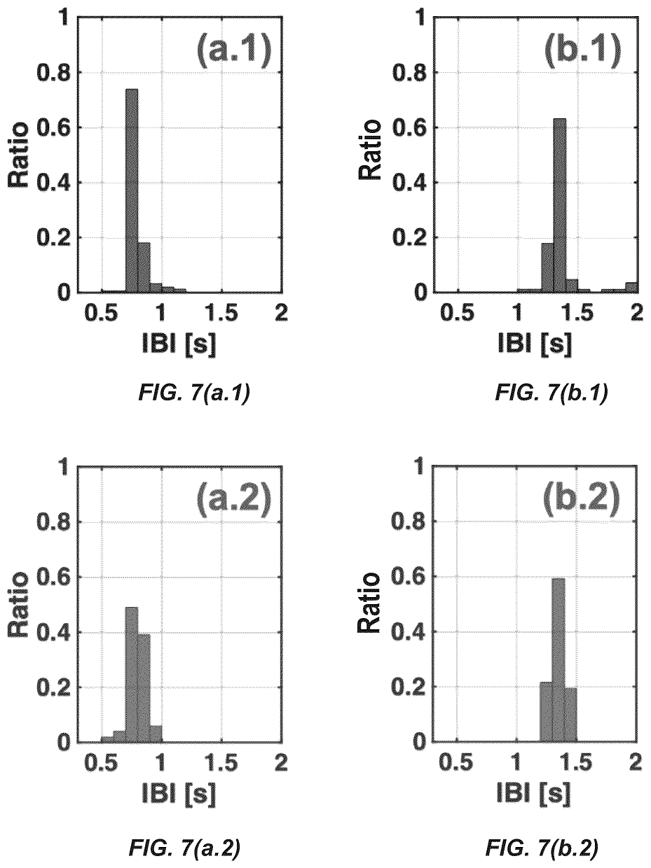

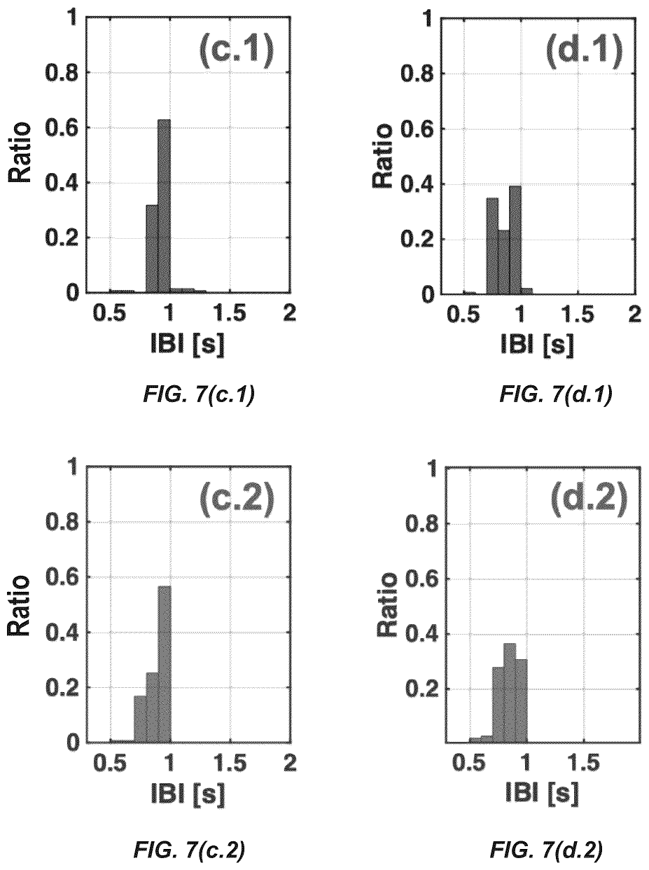

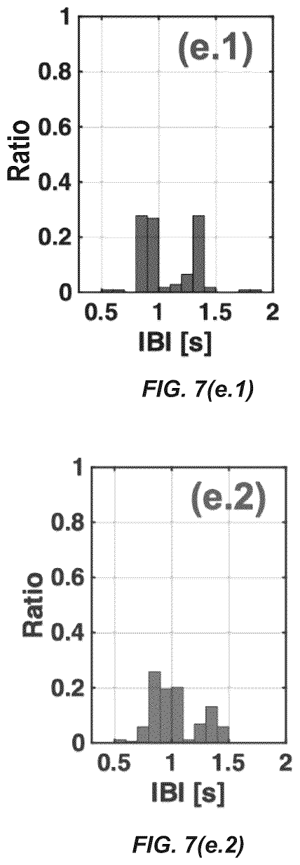

Access to inter-beat-interval (IBI) variations provides valuable information on the cardiovascular system for medical professionals. FIGS. 7(a.1) to 7(e.1) provide plots of inter-beat-interval for five different subjects (randomly selected from the total population of ten subjects) obtained by remote optical depth video processing according to a method disclosed herein, while FIGS. 7(a.2) to 7(e.2) provide plots of inter-beat-interval for the same five subjects as represented in FIGS. 7(a.1) to 7(e.1), but obtained by conventional PPG. These results imply the low HR variability in all subjects except the subject in FIGS. 7(e.1) and 7(e.2). It can be explained by the fact that the subjects were in stationary state. On the other hand, the shapes of the IBI histogram from the five subjects resemble these from the reference. Also, the subject group provides diversity in resting HR, in which the lowest resting HR is about 45 BPM or IBI 0.75 second, while the highest resting HR is about 80 BPM or IBI 1.3 second. The optical depth video measurements were consistent with those obtained by PPG.

### 2) Heart Rate (HR) Accuracy:

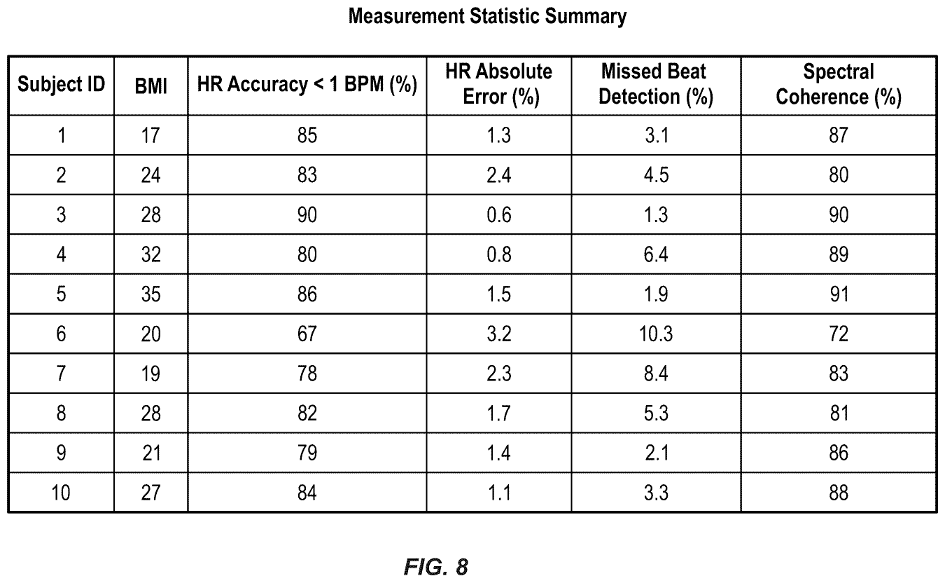

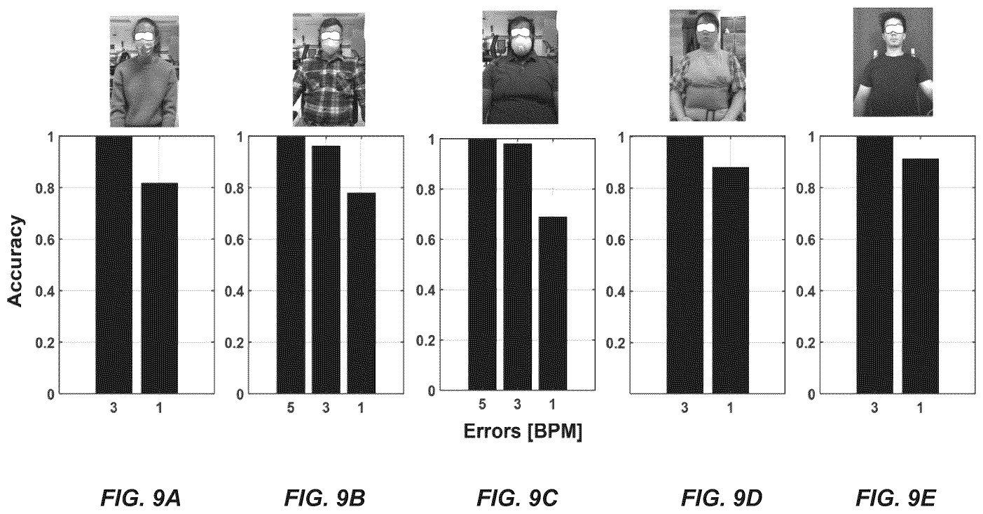

FIG. 8 is a table summarizing measurement statistics including heart rate accuracy, heart rate absolute error, missed beat detection percentage, and spectral coherence percentage for all ten subjects. FIGS. 9A-9E provide bar charts showing heart rate estimation accuracy for the same five subjects from the IBI experiment. The HR accuracy of the above-mentioned five subjects was calculated as the percentage of HR estimates within ($) 5, 3, and 1 BPM, respectively, wherein the depth based HR estimation accuracy achieved 100% within 5 BPM for all five subjects. In particular, for subject 1 (FIG. 9A), subject 4 (FIG. 9D), and subject 5 (FIG. 9E), the HR estimation accuracy attains 100% within 3 BPM. These results again validate the method for cardiac pulse detection disclosed herein. The overall measurement statistics and human subject body mass index (BMI) from the total ten subjects are summarized in FIG. 8. The metrics listed are HR accuracy within 1 BPM tolerance, absolute HR estimation error compared to the ground truth, number of heartbeat peaks missed and spectral coherence measure as introduced previously herein (and defined in Equation (9)). A four-minute dataset for each subject was used to produce the results in FIG. 8. The tabulated values in FIG. 8 were averaged over the estimates processed by a 15 second processing window and a one second stride.

### 3) Simultaneous Detection of Breathing Motion and Heart Rate:

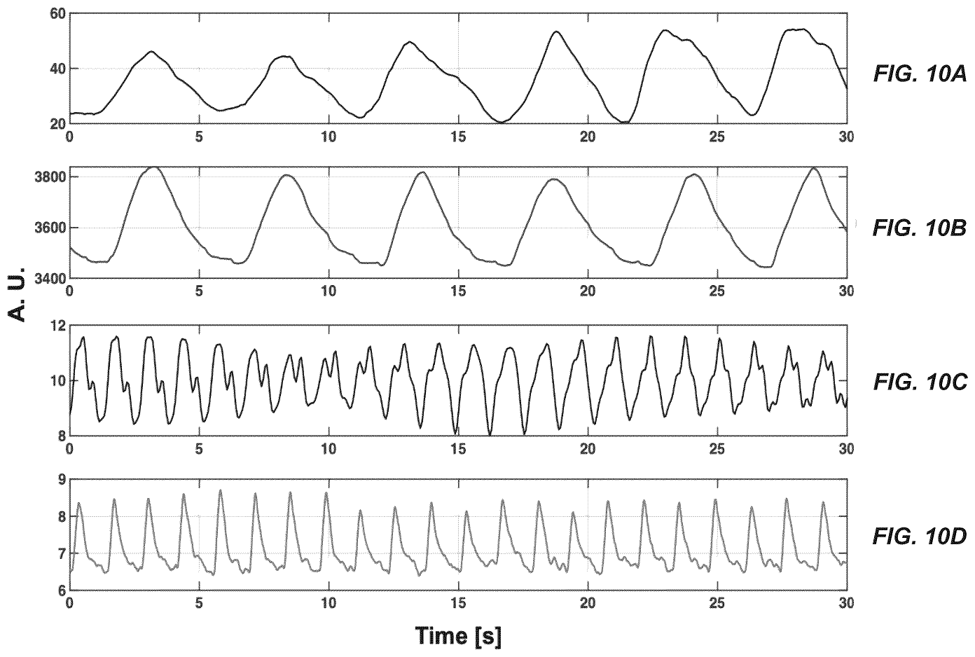

One focus of this disclosure is recovering cardiac pulses over a chest area in the presence of stronger breathing motion, treating breathing as a source of interference to be suppressed. However, simultaneous breathing motion and heartbeat detection is feasible, since breathing is the dominant signal source in the depth measurement. The plots shown in FIGS. 10A-10D depict one representative joint measurement. The estimated breathing pattern shown in FIG. 10A and estimated heartbeat waveform shown in FIG. 10C are compared against the reference signals for breathing pattern in FIG. 10B and for heartbeat waveform shown in FIG. 10D. In particular, FIG. 10A provides a plot of estimated respiration pattern and FIG. 10C provides a plot of recovered heartbeat waveform, both derived from depth signal data as disclosed herein for one subject, whereas FIG. 10B provides a plot of respiration pattern derived from an earlobe photoplethysmography (PPG) sensor and FIG. 10D provides a plot of heart rate from the PPG sensor, with the all measurements represented in FIGS. 10A-10D taken from the same subject over the same interval. Visual inspection shows high-level similarity between the depth measurements and the ground truth.

### 4) Algorithm Performance

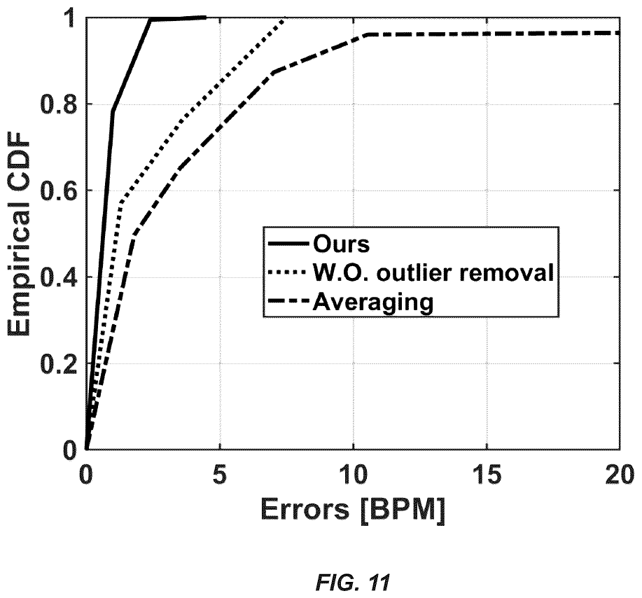

The intermediate HR estimation performance in the processing chain was inspected. The HR estimation performance was evaluated in the form of empirical cumulative distribution functions (Empirical CDFs). An empirical CDF distribution function is the distribution function associated with the empirical measure of a sample (e.g., a step function that increases by 1/n at each of the n data points). FIG. 11 provides plots for empirical cumulative distribution function versus errors for RPCA, for PCA without outlier removal, and for direct averaging of depth pixel data in a region of interest of a subject's chest. The desired curve should approach the top-left corner of FIG. 11. RPCA performs the best, the second best performance was exhibited by regular PCA without removing outliers, and the least favorable performance is obtained by directly averaging the depth pixels in the chest ROI. For example, by fixing the errors at 3 BPM, the corresponding empirical CDF values are 0.93, 0.70 and 0.61. The results show that dividing the region of interest (ROI) into groups and removing outliers are necessary to improve HR accuracy. Naively averaging pixel intensities cannot maintain the HR accuracy.

### (5) Distance to Subject(s)

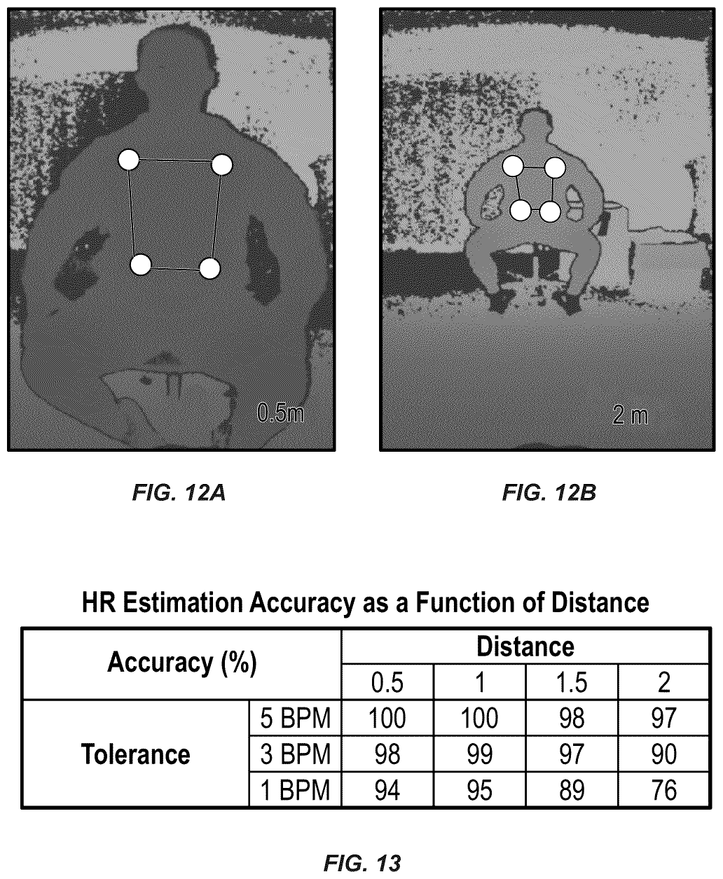

Distance measurements at locations of 0.5, 1, 1.5 and 2 meters were taken from one subject using a depth camera as disclosed herein. FIGS. 12A and 12B provide grayscale-converted depth images of one subject with a superimposed ROI at distances of 0.5 m and 2 m, respectively. Heart rate estimation accuracy is summarized in FIG. 13 at three tolerance levels 1 BPM, 3 BPM and 5 BPM. The heartbeat was detected at all ranges while the accuracy decreased with distance. This is expected because the ranging accuracy in depth video is a function of distance, such that the accuracy decreases with distance. Another reason is that the effective chest ROI reduced with the range as shown in FIGS. 12A-12B, such that the number of pixels available to be processed is also reduced.

### 6) Multiple-Subjects

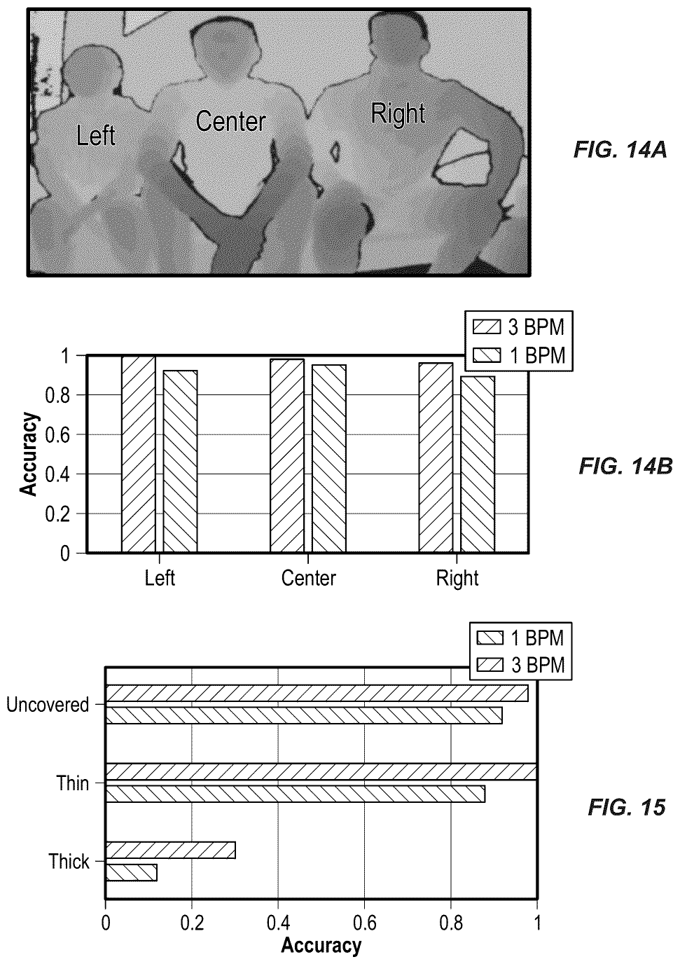

FIG. 14A depicts a scene representing a crowded environment in which three subjects (labeled left, center, and right) were within the field view of the depth sensor. With the help of computer vision techniques, the three subjects were automatically identified. Using the method disclosed herein, HR was obtained simultaneously from all three subjects with high accuracy. The error histogram is displayed in FIG. 14B. One issue in the multi-subject experiment is that the pose estimator failed when the subjects were close to one another. For missed detection, the torso landmarks were manually labeled to generate the results for some trials.

### 7) Clothing Thickness

Clothing may be treated as blockage for observing heartbeat induced motion on the chest. To study the motion sensitivity as a function of the thickness of clothing, depth videos were recorded for three cases, namely: a subject wearing no clothes, a subject wearing a thin layer of clothes, and a subject wearing a thick layer of clothes. The obtained results imply that heartbeat motion sensitivity is significantly reduced when the test subject wore a thick jacket. In particular, the 3 BPM accuracy is reduced by almost 70 percent when thick clothing (versus thin clothing) is worn by a subject to be monitored, as shown in FIG. 15.

### 8) Detection of Peripheral Body Sites (e.g., Head)

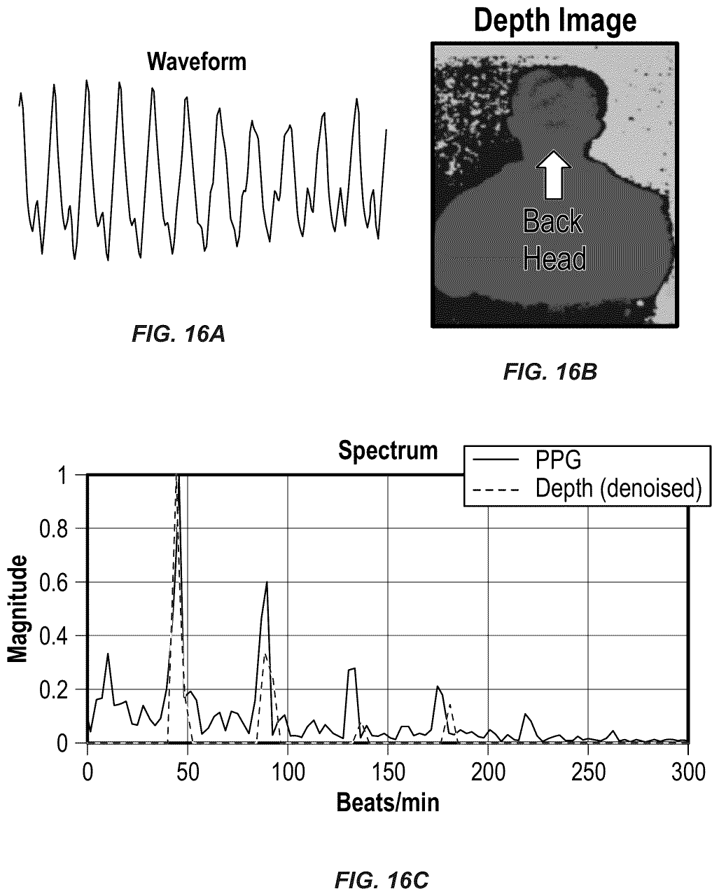

The preceding discussion has been directed primarily on depth sensing focused at a chest area of a subject, but the present disclosure is not so limited. Motion-based systems and methods using depth videos as disclosed herein can be readily extended to other peripheral body sites. To demonstrate the versatility of the proposed method, depth video was recorded from a test subject at the back of the subject's head. Given this measurement site, the signal processing was adapted accordingly. The ROI was selected manually and fixed in the video since no landmarks on the back of the head were previously trained. The rest of the signal processing remained the same. FIG. 16B is a grayscale-converted depth image for an upper rear portion of a subject including the back of the subject's head. FIG. 16A is a cardiac waveform obtained from depth signal data obtained from the back of the head of the subject. FIG. 16C is a cross power spectral density (CPSD) plot of relative magnitude versus heart rate derived from depth signal data obtained from the back of a head of a subject overlaid with a reference PPG spectrum, showing high correlation between the depth signal data peaks and PPG peaks using a method disclosed herein a head of the subject. The spectrum recovered from depth imaging is consistent with the PPG reference and exhibits distinctive spectral energy at the heart rate and its harmonics

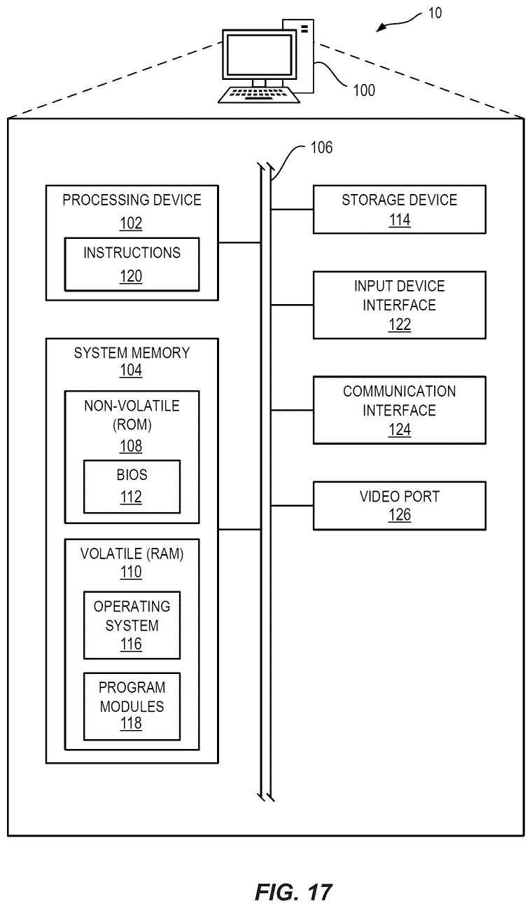

FIG. 17 is a block diagram of the heart rate monitoring system 10 according to embodiments disclosed herein. The heart rate monitoring system 10 includes or is implemented as a computer system 100, which comprises any computing or electronic device capable of including firmware, hardware, and/or executing software instructions that could be used to perform any of the methods or functions described above. In this regard, the computer system 100 may be a circuit or circuits included in an electronic board card, such as a printed circuit board (PCB), a server, a personal computer, a desktop computer, a laptop computer, an array of computers, a personal digital assistant (PDA), a computing pad, a mobile device, or any other device, and may represent, for example, a server or a user's computer.

The exemplary computer system 100 in this embodiment includes a processing device 102 or processor, a system memory 104, and a system bus 106. The processing device 102 represents one or more commercially available or proprietary general-purpose processing devices, such as a microprocessor, central processing unit (CPU), or the like. More particularly, the processing device 102 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 102 is configured to execute processing logic instructions 120 for performing the operations and steps discussed herein.

In this regard, the various illustrative logical blocks, modules, and circuits described in connection with the embodiments disclosed herein may be implemented or performed with the processing device 102, which may be a microprocessor, field programmable gate array (FPGA), a digital signal processor (DSP), an application-specific integrated circuit (ASIC), or other programmable logic device, a discrete gate or transistor logic, discrete hardware components, or any combination thereof designed to perform the functions described herein. Furthermore, the processing device 102 may be a microprocessor, or may be any conventional processor, controller, microcontroller, or state machine. The processing device 102 may also be implemented as a combination of computing devices (e.g., a combination of a DSP and a microprocessor, a plurality of microprocessors, one or more microprocessors in conjunction with a DSP core, or any other such configuration).

The system memory 104 may include non-volatile memory 108 and volatile memory 110. The non-volatile memory 108 may include read-only memory (ROM), erasable programmable read-only memory (EPROM), electrically erasable programmable read-only memory (EEPROM), and the like. The volatile memory 110 generally includes random-access memory (RAM) (e.g., dynamic random-access memory (DRAM), such as synchronous DRAM (SDRAM)). A basic input/output system (BIOS) 112 may be stored in the non-volatile memory 108 and can include the basic routines that help to transfer information between elements within the computer system 100.

The system bus 106 provides an interface for system components including, but not limited to, the system memory 104 and the processing device 102. The system bus 106 may be any of several types of bus structures that may further interconnect to a memory bus (with or without a memory controller), a peripheral bus, and/or a local bus using any of a variety of commercially available bus architectures.

The computer system 100 may further include or be coupled to a non-transitory computer-readable storage medium, such as a storage device 114, which may represent an internal or external hard disk drive (HDD), flash memory, or the like. The storage device 114 and other drives associated with computer-readable media and computer-usable media may provide non-volatile storage of data, data structures, computer-executable instructions, and the like. Although the description of computer-readable media above refers to an HDD, it should be appreciated that other types of media that are readable by a computer, such as optical disks, magnetic cassettes, flash memory cards, cartridges, and the like, may also be used in the operating environment, and, further, that any such media may contain computer-executable instructions for performing novel methods of the disclosed embodiments.

An operating system 116 and any number of program modules 118 or other applications can be stored in the volatile memory 110, wherein the program modules 118 represent a wide array of computer-executable instructions corresponding to programs, applications, functions, and the like that may implement the functionality described herein in whole or in part, such as through instructions 120 on the processing device 102. The program modules 118 may also reside on the storage mechanism provided by the storage device 114. As such, all or a portion of the functionality described herein may be implemented as a computer program product stored on a transitory or non-transitory computer-usable or computer-readable storage medium, such as the storage device 114, volatile memory 110, non-volatile memory 108, instructions 120, and the like. The computer program product includes complex programming instructions, such as complex computer-readable program code, to cause the processing device 102 to carry out the steps necessary to implement the functions described herein.

An operator, such as the user, may also be able to enter one or more configuration commands to the computer system 100 through a keyboard, a pointing device such as a mouse, or a touch-sensitive surface, such as the display device, via an input device interface 122 or remotely through a web interface, terminal program, or the like via a communication interface 124. The communication interface 124 may be wired or wireless and facilitate communications with any number of devices via a communications network in a direct or indirect fashion. An output device, such as a display device, can be coupled to the system bus 106 and driven by a video port 126. Additional inputs and outputs to the computer system 100 may be provided through the system bus 106 as appropriate to implement embodiments described herein.

A novel use of a depth (e.g., ToF) camera to monitor heartbeat by recording chest motion has been demonstrated herein, representing an alternative to existing methods using regular cameras (RGB/NIR). A computer vision model was pre-trained on depth images to track human torso landmarks and create a chest region of interest. Then cardiac pulse information was extracted by applying RPCA to find the most pulsatile signal. A representation in eigen space was developed for signal decomposition and selection.

Those skilled in the art will recognize improvements and modifications to the preferred embodiments of the present disclosure. All such improvements and modifications are considered within the scope of the concepts disclosed herein and the claims that follow. Any of the various features and elements as disclosed herein may be combined with one or more other disclosed features and elements unless indicated to the contrary herein.

What is claimed is:

1. A method for remotely monitoring heart rate of at least one subject, the method comprising:
obtaining optical depth video data of the at least one subject;
identifying a region of interest of the at least one subject from the optical depth video data;
segmenting the region of interest into multiple areas;
identifying pixel intensity with respect to time in the multiple areas to produce a depth signal data matrix including multiple spatial channels;
decomposing the depth signal data matrix into a low-rank spatial-temporal eigenvector matrix to produce refined depth signal data streams; and
projecting the refined depth signal data streams onto a selected pulsatile direction and producing a first cardiac pulse signal for the at least one subject.

2. The method of claim 1, wherein the selected pulsatile direction comprises an optimum pulsatile direction at which cross power spectral density, comprising spectral coherence values as a function of frequency, is maximized.

3. The method of claim 1, further comprising applying bandpass temporal filtering to eliminate excessively high frequency and excessively low frequency data to reduce noise in the first cardiac pulse signal.

4. The method of claim 1, wherein the eigenvector matrix comprises data representing magnitude and direction of each spatial channel contributing to eigenvectors of the eigenvector matrix.

5. The method of claim 1, further comprising detecting a torso area of the at least one subject in the optical depth video data, wherein the identifying of the region of interest comprises removing edge areas from the detected torso area.

6. The method of claim 1, further comprising detecting a head area of the at least one subject in the optical depth video data, wherein the identifying of the region of interest comprises removing edge areas from the detected head area.

7. The method of claim 1, further comprising detecting a respiration rate of the at least one subject.

8. The method of claim 1, wherein the at least one subject comprises a plurality of subjects, and the method comprises producing a different first cardiac pulse signal for each subject of the plurality of subjects.

9. A heart rate monitoring system comprising:
an optical depth sensor; and
an image processor configured to:
receive optical depth video data of at least one subject;
identify a region of interest of the at least one subject from the optical depth video data;
segment the region of interest into multiple areas;
identify pixel intensity with respect to time in the multiple areas to produce a depth signal data matrix including multiple spatial channels;
decompose the depth signal data matrix into a low-rank spatial-temporal eigenvector matrix to produce refined depth signal data streams; and
project the refined depth signal data streams onto a selected pulsatile direction and produce a first cardiac pulse signal for the at least one subject.

10. The system of claim 9, wherein the selected pulsatile direction comprises an optimum pulsatile direction at which cross power spectral density, comprising spectral coherence values as a function of frequency, is maximized.

11. The system of claim 9, wherein the image processor is further configured to apply bandpass temporal filtering to eliminate excessively high frequency and excessively low frequency data to reduce noise in the first cardiac pulse signal.

12. The system of claim 9, wherein the eigenvector matrix comprises data representing magnitude and direction of each spatial channel contributing to eigenvectors of the eigenvector matrix.

13. The system of claim 9, wherein the image processor is further configured to detect a torso area of the at least one subject in the optical depth video data, wherein the identifying of the region of interest comprises removing edge areas from the detected torso area.

14. The system of claim 9, wherein the image processor is further configured to detect a head area of the at least one subject in the optical depth video data, wherein the identifying of the region of interest comprises removing edge areas from the detected head area.

15. The system of claim 9, wherein the image processor is further configured to detect a respiration rate of the at least one subject.

16. The system of claim 9, wherein the at least one subject comprises a plurality of subjects, and the image processor is configured to produce a different first cardiac pulse signal for each subject of the plurality of subjects.

17. A non-transitory computer readable medium comprising computer-readable instructions, that when executed by a processor, cause the processor to perform operations, the operations comprising:
identifying a region of interest of at least one subject from optical depth video data of the at least one subject;
segmenting the region of interest into multiple areas;
identifying pixel intensity with respect to time in the multiple areas to produce a depth signal data matrix including multiple spatial channels;
decomposing the depth signal data matrix into a low-rank spatial-temporal eigenvector matrix to produce refined depth signal data streams; and
projecting the refined depth signal data streams onto a selected pulsatile direction and producing a first cardiac pulse signal for the at least one subject.
